(19) United States
(12) Patent Application Publication
(10) Pub. No.: US 2010/0068268 A1
(43) Pub. Date: Mar. 18, 2010

(54) STARCH-BASED MICROPARTICLES FOR THE RELEASE OF AGENTS DISPOSED THEREIN

(76) Inventors: Miloud Rahmouni, Pierrefonds (CA); Mohamed Nabil Khalid, Montreal (CA); Vinayak Sant, Montreal (CA); Abdelaziz Tafer, Lachenaie (CA); Damon Smith, Saint-Laurent (CA)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/560,046

(22) Filed: Sep. 15, 2009

Related U.S. Application Data
(60) Provisional application No. 61/097,160, filed on Sep. 15, 2008.

(51) Int. Cl. A61K 9/14; A61K 47/36; A61K 9/20; A61K 9/68; A61K 35/66; A61K 35/74; A23L 1/226

(52) U.S. Cl. 424/464; 514/778; 424/499; 514/772.5; 424/48; 424/93.1; 424/93.4; 426/533; 426/271

(57) ABSTRACT

The invention provides starch-based microparticles with high loading capacity for the stabilization and/or controlled release of one or more agents, for example, a pharmaceutical, a taste masking agent, a flavoring agent, or a combination thereof, disposed within the microparticles, and to methods of making and using such microparticles.

### REFERENCE TO RELATED APPLICATIONS

This application claims the benefit of and priority to U.S. provisional patent application Ser. No. 61/097,160, filed Sep. 15, 2008, the entire disclosure of which is incorporated by reference herein.

### FIELD OF THE INVENTION

The invention relates generally to microparticles with a high loading capacity for the stabilization and/or controlled release of one or more agents disposed therein, and more particularly, the invention relates to starch-based microparticles with a high loading capacity for the stabilization and/or controlled release of one or more agents disposed therein.

### BACKGROUND

Over the years, there has been considerable interest in developing compositions that mask the taste and/or odor, stabilize the integrity, and control the release, of one or more agents disposed therein. By masking the taste and/or odor of an agent, such compositions may improve or even make possible the ability of a subject to ingest certain agents. This can have the additional benefit of patient compliance. By stabilizing the agents, the compositions can increase the usable shelf-life of the agent disposed therein. The controlled-release properties of the compositions may reduce the need for frequent administration of the agent while also improving the enjoyment, benefit, efficacy, palatability, tolerability and/or safety of the agent. For example, when the agent is a pharmaceutical, the controlled release properties can result in improved efficacy and safety for the patient by maintaining in vivo drug levels within a therapeutic range, which may not occur when the patient forgets to take or otherwise misses taking one or more doses of an immediate release dosage form.

A variety of controlled release systems have been developed to date. However, there is still an ongoing need for compositions with high loading capacity that mask taste and/or odor while maintaining or enhancing the stability of, and/or permit the controlled release of, agents disposed therein but yet are cost effective and easy to manufacture.

### SUMMARY OF THE INVENTION

In one aspect, the invention provides a method of producing microparticles that have a high loading capacity while enhancing the stability and/or control the release of one or more agents, for example, a pharmaceutical, a flavoring agent, a nutraceutical, an agricultural agent, a cosmetic agent, or a combination thereof, disposed therein. The resulting microparticles, either inherently or by the inclusion of specific components, can also mask the taste and/or odor of one or more of the agents disposed therein.

The method comprises two steps. The first step comprises providing a solution comprising a mixture of from about 5% (w/w) to about 20% (w/w) of cross-linked high amylose starch (for example, from about 7% (w/w) to about 15% (w/w) of cross-linked high amylose starch), and the agent to be released. The second step comprises spray drying the mixture in a spray dryer to produce microparticles having a mean diameter of from about 1 μm to about 200 μm. The agent or agents may be disposed within the lumen of the microparticles and/or the wall of the microparticles. The spray dryer employed preferably has an air inlet temperature in the range of from about 125° C. to about 250° C. and an air outlet temperature in the range of from about 50° C. to about 100° C., or from about 70° C. to about 100° C. The agent can comprise from about 5% to about 50% (w/w), from about 10% to about 45% (w/w), from about 15% to about 40% (w/w), from about 15% to about 35% (w/w), or from about 20% to about 35% (w/w) of the resulting microparticles. It is understood that the microparticles can be hollow.

In another aspect, the invention provides a microparticle composition comprising a plurality of microparticles comprising from about 35% (w/w) to about 70% (w/w) of cross-linked high amylose starch and one or more agents for release from the microparticles. The microparticles have a mean diameter in the range from about 1 μm to about 200 μm. The agent or agents can be disposed within the lumen or the microparticles and/or within the wall or walls of the microparticles. In certain embodiments, the agent comprises from about 5% to about 50% (w/w), from about 10% to about 45% (w/w), from about 15% to about 40% (w/w), from about 15% to about 35% (w/w), or from about 20% to about 35% (w/w) of the resulting microparticles. In certain other embodiments, the microparticles are substantially free of pectin and/or are substantially resistant to degradation by α-amylase.

These and other aspects and features of the invention are described in the following detailed description and claims.

### BRIEF DESCRIPTION OF THE DRAWINGS

The invention is illustrated but is not limited by the annexed drawings, in which

FIG. 1 is a schematic representation of an exemplary procedure for producing the microparticles of the invention;

FIG. 2 is an image taken by a scanning electron microscope of spray dried CONTRAMID® excipient-based microparticles containing 16.5% (w/w) tramadol HCl;

FIG. 3 is an image taken by a scanning electron microscope of a cross-section of a spray dried CONTRAMID® excipient-based microparticle;

FIG. 4 is a graph showing the in vitro release profiles of agents from exemplary microparticles loaded with either 16.5% (w/w) tramadol HCl (--) or caffeine (-▪-);

FIG. 5 is a graph showing the in vitro release profiles of tramadol HCl from microparticles in water (--) or water containing α-amylase (-▴-);

FIG. 6 is an image taken by a scanning electron microscope of spray dried CONTRAMID® excipient-based microspheres containing about 50% (w/w) tramadol HCl;

FIG. 7 is a graph showing the in vitro release profile of tramadol HCl from exemplary microparticles containing about 50% (w/w) tramadol HCl (--);

FIG. 8 is an image taken by a scanning electron microscope of the bacterial strain Lacobacillus rhamnosus; and

FIG. 9 is an image taken by a scanning electron microscope of spray dried CONTRAMID® excipient-based microspheres containing the bacterial strain Lacobacillus rhamnosus.

### DETAILED DESCRIPTION

The invention is based, in part, upon the discovery that it is possible to produce starch-based microparticles with a high loading capacity that provide taste and/or odor masking properties while enhancing the stability and/or permitting the controlled release of one or more agents, for example, a pharmaceutical, a flavoring agent, a taste masking agent, a nutraceutical, a health supplement, a probiotic, an agricultural agent, a cosmetic agent, or a combination thereof, disposed within the microparticles.

I. Microparticles and their Manufacture

The starch-based microparticles are produced by a two step procedure. The first step comprises providing a solution comprising a relatively high concentration of cross-linked high amylose starch (for example, from about 5% (w/w) to about 20% (w/w) of cross-linked high amylose starch, and more particularly, from about 7% (w/w) to about 15% (w/w) of cross-linked high amylose starch) and the agent or agents to be released. The second step comprises spray drying the mixture in a spray dryer to produce microparticles having a mean diameter of from about 1 μm to about 200 μm, from about 5 μm to about 200 μm, from about 5 μm to about 150 μm, from about 1 μm to about 100 μm, from about 10 μm to about 100 μm, from about 1 μm to about 50 μm, from about 1 μm to about 40 μm, from about 1 μm to about 30 μm, from about 2 μm to about 50 μm, from about 3 μm to about 50 μm, from about 4 μm to about 50 μm, or from about 5 μm to about 50 μm. The spray dryer employed preferably has an air inlet temperature in the range of from about 125° C. to about 250° C., and an air outlet temperature in the range of from about 50° C. to about 100° C., or from about 70° C. to about 100° C.

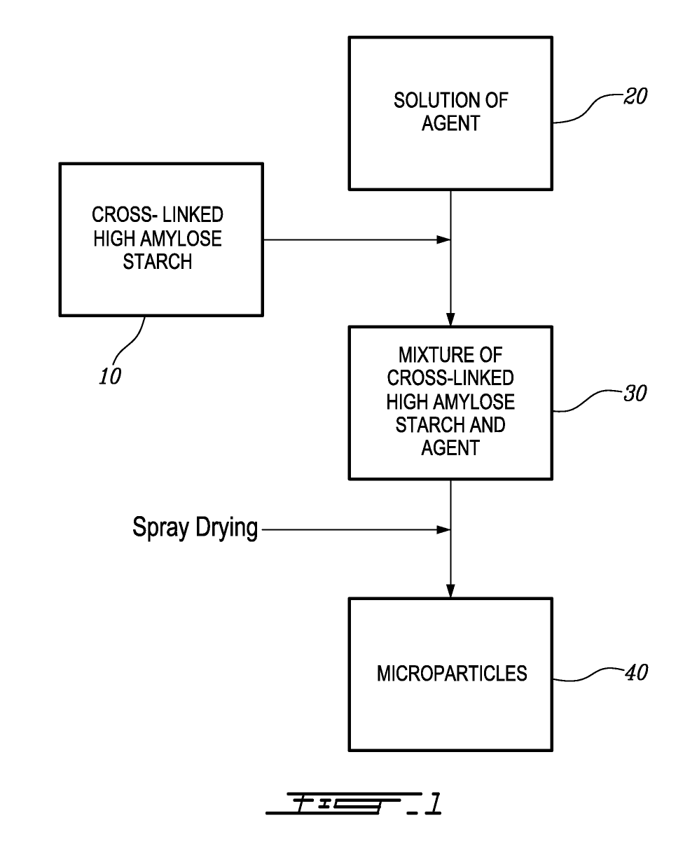

FIG. 1 is a schematic representation of an exemplary protocol for producing the microparticles of the invention. A solution comprising from about 5% (w/w) to about 20% (w/w), or from about 7% (w/w) to about 15% (w/w) of cross-linked high amylose starch 10 is admixed with a solution 20 comprising one or more agents of interest and an optional viscosity reducing agent and an optional dispersing agent, to produce mixture 30 comprising the cross-linked high amylose starch, the agent(s) of interest, and the optional viscosity reducing agent and the optional dispersing agent. The mixture 30 generally is created at a temperature in the range of from about 15° C. to about 70° C., or in the range of from about 30° C. to about 60° C. Mixture 30 then is spray dried to produce a preparation of microparticles 40. Methods for performing spray drying are described, for example, in Giunchedi and Conte (1995) S.T.P. PHARMA SCIENCES 5:276-290; Wendel & Celik (1997) PHARMACEUTICAL TECHNOLOGY 124-156. It is understood, however, that the protocol can be modified, for example, as discussed in more detail below, when the agent of interest is sparingly soluble, slightly soluble, or insoluble in an aqueous solvent, for example, water, an aqueous buffer, water-alcohol mixtures, or aqueous buffer-alcohol mixtures. It is understood that the microparticles can be hollow thereby resulting in hollow microparticles.

When the agent or agents to be included in the microspheres include agents that are poorly soluble or insoluble in water (for example, menthol (see, Example 3) and vanillin (see, Example 4), the agents can be heated to form a dispersion and then are combined with cross-linked high amylose starch 10 to produce a mixture 30 that is an emulsion. The emulsion can then be spray dried to produce a preparation of microparticles 40.

A variety of cross-linked high amylose starches may be used in the practice of the invention. The cross-linking of high amylose starch can be produced using procedures described in the art. For example, cross-linking of amylose can be carried out in the manner described in Mateescu [BIOCHEMIE 60: 535-537 (1978)] by reacting amylose with epichlorohydrin in an alkaline medium. In the same manner, starch can also be cross-linked with a reagent selected from the group consisting of epichlorohydrin, adipic acid anhydride, sodium trimetaphosphate and phosphorous oxychloride or other cross-linking agents including, but not limited to, 2,3-dibromopropanol, linear mixed anhydrides of acetic and di- or tribasic carboxylic acids, vinyl sulfone, diepoxides, cyanuric chloride, hexahydro-1,3,5-trisacryloyl-s-triazine, hexamethylene diisocyanate, toluene 2,4-diisocyanate, N,N-methylenebisacrylamide, N,N′-bis(hydroxymethyl)ethyleneurea, mixed carbonic-carboxylic acid anhydrides, imidazolides of carbonic and polybasic carboxylic acids, imidazolium salts of polybasic carboxylic acids, and guanidine derivatives of polycarboxylic acids. The reaction conditions employed will vary with the type and amount of the cross-linking agent that is used, as well as the base concentration, amount and type of starch. In some embodiments, the cross-linked high amylose starch may be gelatinized after cross-linking.

It is contemplated that starches containing more than about 40% w/w amylose can be used to form cross-linked high amylose starch, e.g., pea and wrinkled pea starch, bean starch, hybrids or genetically modified tapioca or potato starch, or any other root, tuber or cereal starch. Preferably, high amylose starch containing about 70% w/w amylose is used as the base material. For example, high amylose starch, Cerestar AmyloGel 03003 (Cerestar U.S.A. Inc.) can be used. In certain formulations, the excipient comprises cross-linked high amylose starch comprising between about 65% and about 75% by weight of amylose cross-linked with phosphorus oxychloride.

In one embodiment, the cross-linked high amylose starch is cross-linked with phosphorus oxychloride and/or comprises hydroxypropyl side chains. Exemplary cross-linked high amylose starch has been developed by and is available commercially from Labopharm, Inc., Laval, Canada, under the tradename CONTRAMID®. The synthesis of the CONTRAMID® excipient is described, for example, in U.S. Pat. No. 6,607,748.

In certain embodiments, when the agent is mixed with the cross-linked high amylose starch, a high shear mixer can be used to reduce the viscosity of the resulting mixture. The high shear mixer can reduce the mixture to uniform-sized molecules through maceration, cutting and blending, thus reducing the thickness as well as the viscosity of the solution. Suitable high shear mixers include, for example, the MP550 Turbo from Robot® Coupe (Jackson, Miss.) and the IKA Ultra-Turrax T-25 Basic S1 from IKA Works (Wilminton, N.C.) and are used in accordance with the manufacturer's instructions.

Alternatively, the appropriate viscosity of the mixture can be achieved by mixing the cross-linked high amylose starch with a viscosity reducing agent, either with or without high shear mixing. The viscosity reducing agent can be selected from the group consisting of a water soluble linear polymer, a water soluble linear copolymer, and a polyol. In certain embodiments, the viscosity reducing agent is selected from the group consisting of a polyvinylpyrrolidone-vinyl acetate copolymer, polyvinyl pyrrolidone, propylene glycol, maltodextrin, sorbitol, and mannitol. In a preferred embodiment, the viscosity reducing agent is polyvinylpyrrolidone-vinyl acetate copolymer, also sold under the trade name KOLLIDON VA 64 Fine from BASF, New Jersey.

In certain embodiments, in the liquid mixture prior to or in the resulting composition after spray drying, the ratio of the cross-linked high amylose starch to the optional viscosity reducing agent is from about 80:20 (w/w) to about 40:60 (w/w), for example, 80:20 (w/w), 75:25 (w/w), 70:30 (w/w), 65:35 (w/w), 60:40 (w/w), 55:45 (w/w), 50:50 (w/w), 45:55 (w/w), or 40:60 (w/w). In certain embodiments, the ratio of the cross-linked high amylose starch to the optional viscosity reducing agent is about 60:40 (w/w). In certain embodiments, the mixture prior to or after spray drying is substantially free of pectin, i.e., has less than about 3% (w/w), less than about 2% (w/w) or less than about 1% (w/w) pectin.

In certain embodiments, the agent is soluble in an aqueous solvent (e.g., 1 gram of agent dissolves in less than 1 mL or up to about 30 mL of aqueous solvent), or the agent is sparingly soluble in an aqueous solvent (e.g., 1 gram of agent dissolves in greater than 30 mL to about 100 mL of aqueous solvent), or the agent is slightly soluble in aqueous solvent (e.g., 1 gram of agent dissolves in from about 100 mL to about 10,000 mL of aqueous solvent), or the agent is insoluble in an aqueous solvent (e.g., 1 gram of agent dissolves in greater than 10,000 mL of aqueous solvent). The method, however, can be modified as described below depending upon the melting point of the agent. For example, if the agent has a low melting point (e.g., below 60° C.), the agent can be melted by heating and then added to the cross-linked high amylose starch to form an emulsion. Alternatively, if the agent has a high melting point (e.g., above 60° C.), the agent can be combined with a dispersing agent and then added to the cross-linked high amylose starch to form an oily emulsion. In either approach, an optional surface active agent, such as Tween® (for example, Tween®20 or Tween® 80), can be added to the emulsion in order to improve the stability of the emulsion.

Exemplary dispersing agents include, for example, oils, for example, mineral oil and vegetable oils, including for example canola oil, corn oil, cottonseed oil, coconut oil, cocoa butter, grapeseed oil, hemp oil, olive oil, palm oil, peanut oil, rapeseed oil, safflower oil, sesame oil, soybean oil, sunflower oil and the like. Other exemplary dispersing agents include surfactants and emulsifiers, for example, alginates, carageenan, xanthan and other carbohydrate gums, modified starches, cellulose derivatives, fatty acids and their salts, glycerides and esters thereof, sorbitan esters, polysorbates (Tween®, for example, Tween® 20 or Tween® 80), polyoxyethylene stearates, poloxamers, sodium dodecyl sulphate, and the like.

The spray dried microparticle compositions described herein comprise a plurality of microparticles comprising from about 35% (w/w) to about 70% (w/w) of cross-linked high amylose starch and an agent for release from the microparticles, wherein the microparticles have a mean diameter in the range from about 1 μm to about 200 μm, from about 5 μm to about 200 μm, from about 5 μm to about 150 μm, from about 1 μm to about 100 μm, from about 10 μm to about 100 μm, from about 1 μm to about 50 μm, from about 1 μm to about 40 μm, from about 1 μm to about 30 μm, or from about 2 μm to about 50 μm, from about 3 μm to about 50 μm, from about 4 μm to about 50 μm, or from about 5 μm to about 50 μm. Depending upon the circumstances, it can be desirable to include an optional viscosity reducing agent and/or dispersing agent in the microparticles. The microparticles, in certain embodiments, are substantially free of pectin and/or are substantially resistant to degradation by α-amylase (for example, the release of the agent from the microparticles in water containing 5000 Units/L of α-amylase is within 20%, more preferably within 10% of the release of the agent in water in the absence of α-amylase).

The microparticles described herein can be disposed within a capsule, tablet, caplet, chewing gum, film, wafer or an orally disintegrating tablet. Such formulations can be orally administered to an individual in need of such an agent.

II Agents Incorporated into the Microparticles

It is understood that the agent incorporated into the microparticles can be a pharmaceutical, a taste masking agent, a flavoring agent, a nutraceutical (for example, a botanical or herbal extract, a health supplement, or a weight loss agent), a health supplement, a probiotic agent, a cosmetic agent, an agricultural agent, or a combination thereof.

For example, it is contemplated that the microparticles can contain (i) a pharmaceutical, (ii) a pharmaceutical and a taste masking agent, (iii) a pharmaceutical and a flavoring agent, and (iv) a pharmaceutical, a taste masking a agent and a flavoring agent. Furthermore, it is contemplated that the microparticles can comprise a plurality of different pharmaceuticals and optionally a taste masking agent and/or a flavoring agent. It is understood that in certain embodiments, based on the fact that the microparticles have controlled release properties, the microparticles inherently have taste masking properties because the pharmaceutical is released in the mouth at a rate so that any taste is reduced or eliminated. Furthermore, it is understood that in each of the foregoing formulations, the pharmaceutical can be replaced by or supplemented with one or more of a nutraceutical, a health supplement, a probiotic agent, a cosmetic agent or an agricultural agent.

The microparticles produced by the methods described herein have a high loading capacity for the agent. In certain embodiments, the agent or agents incorporated into the microparticles comprise from about 5% to about 50% (w/w) of the microparticles, for example, from about 10% to about 50% (w/w), from about 20% to about 50% (w/w), from about 30% to about 50% (w/w), from about 40% to about 50% (w/w), from about 5% to about 40% (w/w), from about 5% to about 30% (w/w), from about 5% to about 20% (w/w), from about 10% to about 45% (w/w), from about 10% to about 40% (w/w), from about 10% to about 30% (w/w), from about 10% to about 20% (w/w), from about 15% to about 40% (w/w), from about 15% to about 35% (w/w), from about 15% to about 30% (w/w), from about 20% to about 35% (w/w), or from about 20% to about 30% (w/w) of the microparticles.

The loading capacity of the microparticles can be determined using a number of protocols known in the art. In one exemplary approach, a fraction of the microparticles of interest are weighed and transferred to a 100 mL volumetric flask. Then, depending upon the physical properties of the agent, the agent is extracted using an appropriate solvent. For example, for a water soluble agent, for example, tramadol, 60 mL of a water-acetonitrite mixture (40:60 (v/v)) is added to the flask. Following shaking for 1-5 minutes and then sonication using a Branson 8510 (Danbury, Conn.) sonic bath for 10 to about 30 minutes, the solution is permitted to equilibrate for 10 to about 30 minutes. The solution then is made up to 100 mL with the water-acetonitrite mixture. Then 5 mL of the resulting solution is diluted 20-fold with water-acetonitrite (77:23 (v/v)). After filtration to remove particles (for example, via filtering through a 0.45 μm PTFE filter), the diluent is analyzed by high pressure liquid chromatography (HPLC). Based on the results from the HPLC analysis, it is possible to determine the amount (percent w/w) of agent in the fraction of microparticles analyzed. It is understood, however, that the actual protocol and reagents (e.g., solvents) may vary depending upon what agent or agents are incorporated into the microparticles.

A—Pharmaceuticals

The microparticles described herein are particularly useful in the delivery of pharmaceuticals.

The terms “pharmaceutical,” “pharmaceutically active agent,” and “active pharmaceutical ingredient” are used interchangeably herein, and refer to any chemical moiety that is a biologically, physiologically, or pharmacologically active substance that acts locally or systemically in a subject, and includes pharmaceutically acceptable salts, esters and prodrugs thereof. It is understood that the pharmaceutically active agent can be a small molecule, synthetic molecule, or biologic, for example, a protein, peptide, glycoprotein, or nucleic acid. Exemplary pharmaceuticals are described in well-known literature references such as the Merck Index, the Physicians Desk Reference, and The Pharmacological Basis of Therapeutics, and include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

Compositions and formulations contemplated herein may include one or more pharmaceuticals. For example, a composition may include two, three or more different pharmaceuticals.

The pharmaceuticals can vary widely with the purpose for the composition. It is contemplated that one or a plurality of different pharmaceuticals are included in the microparticles described herein. Non-limiting examples of broad categories of useful pharmaceuticals include the following therapeutic categories: anabolic agents, antacids, anti-asthmatic agents, anti-cholesterolemic and anti-lipid agents, anti-coagulants, anti-convulsants, anti-diarrheals, anti-emetics, anti-infective agents, anti-inflammatory agents, anti-manic agents, anti-nauseants, anti-neoplastic agents, anti-obesity agents, anti-pyretic and analgesic agents, anti-spasmodic agents, anti-thrombotic agents, anti-uricemic agents, anti-anginal agents, antihistamines, anti-tussives, appetite suppressants, biologicals, cerebral dilators, coronary dilators, decongestants, diuretics, diagnostic agents, erythropoietic agents, expectorants, gastrointestinal sedatives, hyperglycemic agents, hypnotics, hypoglycemic agents, ion exchange resins, laxatives, mineral supplements, mucolytic agents, neuromuscular drugs, peripheral vasodilators, psychotropics, sedatives, stimulants, thyroid and anti-thyroid agents, uterine relaxants, vitamins, and pro-drugs.

More specifically, non-limiting examples of useful pharmaceutically active substances include the following therapeutic categories: analgesics, such as, nonsteroidal anti-inflammatory drugs, opiate agonists and salicylates; antihistamines, such as, H₁-blockers and H₂-blockers; anti-infective agents, such as, anthelmintics, antianaerobics, antibiotics, aminoglycoside antibiotics, antifungal antibiotics, cephalosporin antibiotics, macrolide antibiotics, miscellaneous β-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, antiretroviral agents, scabicides, and urinary anti-infectives; antineoplastic agents, such as alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics; autonomic agents, such as, anticholinergics, antimuscarinic anticholinergics, ergot alkaloids, parasympathomimetics, cholinergic agonist parasympathomimetics, cholinesterase inhibitor parasympathomimetics, sympatholytics, α-blocker sympatholytics, β-blocker sympatholytics, sympathomimetics, and adrenergic agonist sympathomimetics; cardiovascular agents, such as, antianginals, β-blocker antianginals, calcium-channel blocker antianginals, nitrate antianginals, antiarrhythmics, cardiac glycoside antiarrhythmics, class I antiarrhythmics, class II antiarrhythmics, class III antiarrhythmics, class IV antiarrhythmics, antihypertensive agents, α-blocker antihypertensives, angiotensin-converting enzyme inhibitor (ACE inhibitor) antihypertensives, β-blocker antihypertensives, calcium-channel blocker antihypertensives, central-acting adrenergic antihypertensives, diuretic antihypertensive agents, peripheral vasodilator antihypertensives, antilipemics, bile acid sequestrant antilipemics, HMG-COA reductase inhibitors, inotropes, cardiac glycoside inotropes, and thrombolytic agents; dermatological agents, such as, antihistamines, anti-inflammatory agents, corticosteroid anti-inflammatory agents; electrolytic and renal agents, such as, acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as, pancreatic enzymes and thrombolytic enzymes; gastrointestinal agents, such as, antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H₂-blocker anti-ulcer agents, cholelitholytic agents, digestants, emetics, laxatives and stool softeners, and prokinetic agents; hematological agents, such as, antianemia agents, hematopoietic antianemia agents, coagulation agents, anticoagulants, hemostatic coagulation agents, platelet inhibitor coagulation agents, thrombolytic enzyme coagulation agents, and plasma volume expanders; hormones and hormone modifiers, such as, abortifacients, adrenal agents, corticosteroid adrenal agents, androgens, anti-androgens, antidiabetic agents, sulfonylurea antidiabetic agents, antihypoglycemic agents, oral contraceptives, progestin contraceptives, estrogens, fertility agents, oxytocics, parathyroid agents, pituitary hormones, progestins, antithyroid agents, thyroid hormones, and tocolytics; immunobiologic agents, such as, immunoglobulins, immunosuppressives, toxoids, and vaccines; local anesthetics, such as, amide local anesthetics and ester local anesthetics; musculoskeletal agents, such as, anti-gout anti-inflammatory agents, corticosteroid anti-inflammatory agents, gold compound anti-inflammatory agents, immuno-suppressive anti-inflammatory agents, nonsteroidal anti-inflammatory drugs (NSAIDs), salicylate anti-inflammatory agents, skeletal muscle relaxants, neuromuscular blocker skeletal muscle relaxants, and reverse neuromuscular blocker skeletal muscle relaxants; neurological agents, such as, anticonvulsants, barbiturate anticonvulsants, benzodiazepine anticonvulsants, anti-migraine agents, anti-parkinsonian agents, anti-vertigo agents, opiate agonists, and opiate antagonists; psychotropic agents, such as, antidepressants, heterocyclic antidepressants, monoamine oxidase inhibitors (MAOIs), selective serotonin re-uptake inhibitors (SSRIs), tricyclic antidepressants, antimanics, antipsychotics, phenothiazine antipsychotics, anxiolytics, sedatives, and hypnotics, barbiturate sedatives and hypnotics, benzodiazepine anxiolytics, sedatives, and hypnotics, and psychostimulants; respiratory agents, such as, antitussives, bronchodilators, adrenergic agonist bronchodilators, antimuscarinic bronchodilators, expectorants, mucolytic agents, respiratory anti-inflammatory agents, and respiratory corticosteroid anti-inflammatory agents; toxicology agents, such as, antidotes, heavy metal antagonists/chelating agents, substance abuse agents, deterrent substance abuse agents, and withdrawal substance abuse agents; minerals; and vitamins, such as, vitamin A, vitamin B, vitamin C, vitamin D, vitamin E, and vitamin K.

Preferred classes of useful pharmaceuticals from the above categories include: (1) nonsteroidal anti-inflammatory drugs (NSAIDs) analgesics, such as, diclofenac, ibuprofen, ketoprofen, and naproxen; (2) opiate agonist analgesics, such as, codeine, fentanyl, tramadol, hydromorphone, hydrocodone, oxycodone, oxymorphone and morphine; (3) salicylate analgesics, such as, aspirin; (4) H1-blocker antihistamines, such as, clemastine and terfenadine; (5) H2-blocker antihistamines, such as, cimetidine, famotidine, nizadine, and ranitidine; (6) anti-infective agents, such as, mupirocin; (7) antianaerobic anti-infectives, such as, chloramphenicol and clindamycin; (8) antifungal antibiotic anti-infectives, such as, amphotericin b, clotrimazole, fluconazole, and ketoconazole; (9) macrolide antibiotic anti-infectives, such as, azithromycin and erythromycin; (10) miscellaneous β-lactam antibiotic anti-infectives, such as, aztreonam and imipenem; (11) penicillin antibiotic anti-infectives, such as, nafcillin, oxacillin, penicillin G, and penicillin V; (12) quinolone antibiotic anti-infectives, such as, ciprofloxacin and norfloxacin; (13) tetracycline antibiotic anti-infectives, such as, doxycycline, minocycline, and tetracycline; (14) antituberculosis antimycobacterial anti-infectives such as, isoniazid (INH), and rifampin; (15) antiprotozoal anti-infectives, such as, atovaquone and dapsone; (16) antimalarial antiprotozoal anti-infectives, such as, chloroquine and pyrimethamine; (17) anti-retroviral anti-infectives, such as, ritonavir and zidovudine; (18) antiviral anti-infective agents, such as, acyclovir, ganciclovir, interferon alfa, and rimantadine; (19) alkylating antineoplastic agents, such as, carboplatin and cisplatin; (20) nitrosourea alkylating antineoplastic agents, such as, carmustine (BCNU); (21) antimetabolite antineoplastic agents, such as, methotrexate; (22) pyrimidine analog antimetabolite antineoplastic agents, such as, fluorouracil (5-FU) and gemcitabine; (23) hormonal antineoplastics, such as, goserelin, leuprolide, and tamoxifen; (24) natural antineoplastics, such as, aldesleukin, interleukin-2, docetaxel, etoposide (VP-16), interferon alfa, paclitaxel, and tretinoin (ATRA); (25) antibiotic natural antineoplastics, such as, bleomycin, dactinomycin, daunorubicin, doxorubicin, and mitomycin; (26) vinca alkaloid natural antineoplastics, such as, vinblastine and vincristine; (27) autonomic agents, such as, nicotine; (28) anticholinergic autonomic agents, such as, benztropine and trihexyphenidyl; (29) antimuscarinic anticholinergic autonomic agents, such as, atropine and oxybutynin; (30) ergot alkaloid autonomic agents, such as, bromocriptine; (31) cholinergic agonist parasympathomimetics, such as, pilocarpine; (32) cholinesterase inhibitor parasympathomimetics, such as, pyridostigmine; (33) α-blocker sympatholytics, such as, prazosin; (34) 9-blocker sympatholytics, such as, atenolol; (35) adrenergic agonist sympathomimetics, such as, albuterol and dobutamine; (36) cardiovascular agents, such as, aspirin; (37) i-blocker antianginals, such as, atenolol and propranolol; (38) calcium-channel blocker antianginals, such as, nifedipine and verapamil; (39) nitrate antianginals, such as, isosorbide dinitrate (ISDN); (40) cardiac glycoside antiarrhythmics, such as, digoxin; (41) class I antiarrhythmics, such as, lidocaine, mexiletine, phenyloin, procainamide, and quinidine; (42) class II antiarrhythmics, such as, atenolol, metoprolol, propranolol, and timolol; (43) class III antiarrhythmics, such as, amiodarone; (44) class IV antiarrhythmics, such as, diltiazem and verapamil; (45) a blocker antihypertensives, such as, prazosin; (46) angiotensin-converting enzyme inhibitor (ACE inhibitor) antihypertensives, such as, captopril and enalapril; (47) β-blocker antihypertensives, such as, atenolol, metoprolol, nadolol, and propanolol; (48) calcium-channel blocker antihypertensive agents, such as, diltiazem and nifedipine; (49) central-acting adrenergic antihypertensives, such as, clonidine and methyldopa; (50) diurectic antihypertensive agents, such as, amiloride, furosemide, hydrochlorothiazide (HCTZ), and spironolactone; (51) peripheral vasodilator antihypertensives, such as, hydralazine and minoxidil; (52) antilipemics, such as, gemfibrozil and probucol; (53) bile acid sequestrant antilipemics, such as, cholestyramine; (54) HMG-CoA reductase inhibitor antilipemics, such as, lovastatin and pravastatin; (55) inotropes, such as, aminone, dobutamine, and dopamine; (56) cardiac glycoside inotropes, such as, digoxin; (57) thrombolytic agents, such as, alteplase (TPA), anistreplase, streptokinase, and urokinase; (58) dermatological agents, such as, colchicine, isotretinoin, methotrexate, minoxidil, tretinoin (ATRA); (59) dermatological corticosteroid anti-inflammatory agents, such as, betamethasone and dexamethasone; (60) antifungal anti-infectives, such as, amphotericin B, clotrimazole, miconazole, and nystatin; (61) antiviral anti-infectives, such as, acyclovir; (62) antineoplastics, such as, fluorouracil (5-FU); (63) electrolytic and renal agents, such as, lactulose; (64) loop diuretics, such as, furosemide; (65) potassium-sparing diuretics, such as, triamterene; (66) thiazide diuretics, such as, hydrochlorothiazide (HCTZ); (67) uricosuric agents, such as, probenecid; (68) enzymes, such as, RNase and DNase; (69) thrombolytic enzymes, such as, alteplase, anistreplase, streptokinase and urokinase; (70) antiemetics, such as, prochlorperazine; (71) salicylate gastrointestinal anti-inflammatory agents, such as, sulfasalazine; (72) gastric acid-pump inhibitor anti-ulcer agents, such as, omeprazole; (73) H₂-blocker anti-ulcer agents, such as, cimetidine, famotidine, nizatidine, and ranitidine; (74) digestants, such as, pancrelipase; (75) prokinetic agents, such as, erythromycin; (76) fentanyl; (77) hematopoietic antianemia agents, such as, erythropoietin, filgrastim (G-CSF), and sargramostim (GM-CSF); (78) coagulation agents, such as, antihemophilic factors 1-10 (AHF 1-10); (79) anticoagulants, such as, warfarin; (80) thrombolytic enzyme coagulation agents, such as, alteplase, anistreplase, streptokinase and urokinase; (81) hormones and hormone modifiers, such as, bromocriptine; (82) abortifacients, such as, methotrexate; (83) antidiabetic agents, such as, insulin; (84) oral contraceptives, such as, estrogen and progestin; (85) progestin contraceptives, such as, levonorgestrel and norgestrel; (86) estrogens, such as, conjugated estrogens, diethylstilbestrol (DES), estrogen (estradiol, estrone, and estropipate); (87) fertility agents, such as clomiphene, human chorionic gonadatropin (HCG), and menotropins; (88) parathyroid agents, such as, calcitonin; (89) pituitary hormones, such as, desmopressin, goserelin, oxytocin, and vasopressin (ADH); (90) progestins, such as, medroxyprogesterone, norethindrone, and progesterone; (91) thyroid hormones, such as, levothyroxine; (92) immunobiologic agents, such as, interferon beta-1b and interferon gamma-1b; (93) immunoglobulins, such as, immune globulin 1M, IMIG, IGIM and immune globulin IV, IVIG, IGIV; (94) amide local anesthetics, such as, lidocaine; (95) ester local anesthetics, such as benzocaine and procaine; (96) musculoskeletal corticosteroid anti-inflammatory agents, such as, beclomethasone, betamethasone, cortisone, dexamethasone, hydrocortisone, and prednisone; (97) musculoskeletal anti-inflammatory immunosuppressives, such as, azathioprine, cyclophosphamide, and methotrexate; (98) musculoskeletal nonsteroidal anti-inflammatory drugs (NSAIDs), such as, diclofenac, ibuprofen, ketoprofen, ketorolac, and naproxen; (99) skeletal muscle relaxants, such as, baclofen, cyclobenzaprine, and diazepam; (100) reverse neuromuscular blocker skeletal muscle relaxants, such as, pyridostigmine; (101) neurological agents, such as, nimodipine, riluzole, tacrine, trazodone, and ticlopidine; (102) anticonvulsants, such as, carbamazepine, gabapentin, lamotrigine, phenyloin, and valproic acid; (103) barbiturate anticonvulsants, such as, phenobarbital and primidone; (104) benzodiazepine anticonvulsants, such as, clonazepam, diazepam, and lorazepam; (105) anti-parkinsonian agents, such as, bromocriptine, levodopa, carbidopa, and pergolide; (106) anti-vertigo agents, such as, meclizine; (107) opiate agonists, such as, codeine, fentanyl, hydromorphone, methadone, tramadol, and morphine; (108) opiate antagonists, such as, naloxone; (109) β-blocker anti-glaucoma agents, such as, timolol; (110) miotic anti-glaucoma agents, such as, pilocarpine; (111) ophthalmic aminoglycoside antiinfectives, such as, gentamicin, neomycin, and tobramycin; (112) ophthalmic quinolone anti-infectives, such as, ciprofloxacin, norfloxacin, and ofloxacin; (113) ophthalmic cortico steroid anti-inflammatory agents, such as, dexamethasone and prednisolone; (114) ophthalmic nonsteroidal anti-inflammatory drugs (NSAIDs), such as, diclofenac; (115) antipsychotics, such as, clozapine, haloperidol, and risperidone; (116) benzodiazepine anxiolytics, sedatives and hypnotics, such as, alprazolam, clonazepam, diazepam, lorazepam, oxazepam, and prazepam; (117) psychostimulants, such as, methylphenidate and pemoline; (118) antitussives, such as, codeine; (119) bronchodilators, such as, theophylline; (120) adrenergic agonist bronchodilators, such as, albuterol; (121) respiratory corticosteroid anti-inflammatory agents, such as, dexamethasone; (122) antidotes, such as, flumazenil and naloxone; (123) heavy metal antagonists/chelating agents, such as, penicillamine; (124) deterrent substance abuse agents, such as, disulfuram, naltrexone, and nicotine; (125) withdrawal substance abuse agents, such as, bromocriptine; (126) minerals, such as, iron, calcium, and magnesium; (127) vitamin B compounds, such as, cyanocobalamin (vitamin B₁₂) and niacin (vitamin B₃); (128) vitamin C compounds, such as, ascorbic acid; (129) vitamin D compounds, such as, calcitriol, and (130) histamine type drugs, such as, betahistine hydrochloride.

B—Taste Masking Agents

The microparticles, when desired, can also contain one or more taste masking agents. Exemplary taste masking agents include, without limitation, (1) sugar alcohols, for example, isomalt, maltitol, sorbitol, xylitol, mannitol, erythritol, lactitol, and glycerol (2) sugars, for example, sucrose, glucose (corn syrup), dextrose, invert sugar, fructose, and polydextrose, (3) cellulose, (4) saccharine and its various salts, such as, the sodium salt or the calcium salt; (5) cyclamic acid and its various salts, such as, the sodium salt, (6) dipeptide sweeteners, for example, aspartame, alitame, and neotame, (7) dihydrochalcone compounds, (8) glycyrrhizin, (9) stevia Rebaudiana (Stevioside), (10) thaumatin, (11) dihydroflavinol, (12) hydroxyguaiacol esters, (13) L-amino dicarboxylic acid gem-diamines, (14) L-aminodicarboxylic acid aminoalkenoic acid ester amides, and (15) synthetic sweeteners, for example, acesulfame potassium, sucralose, 3,6-dihydro-6-methyl-1-1,2,3-oxathiazin-4-one-2,2-dioxide, and salts thereof.

C—Flavoring Agents

The microparticles, when desired, can also contain one or more flavoring agents. Exemplary flavoring agents include, without limitation, (1) natural and synthetic flavoring agents, for example, mints (for example, peppermint and spearmint), menthol, artificial vanilla, vanillin, ethyl vanillin, cinnamon, cinnamyl alcohol, methyl cinnamate, ethyl cinamate, methyl benzoate, clove, chlorophyll, eucalyptus oil, ginger, licorice, copper gluconate (retsyn), thymol, and wintergreen, (2) citrus oils including lemon, orange, lime, grapefruit, yazu, sudachi, and (3) fruit essences including apple, pear, peach, grape, blueberry, strawberry, raspberry, cherry, plum, pineapple, banana, melon, apricot, ume, blackberry, tropical fruit, mango, mangosteen, pomegranate, papaya, watermelon, and so forth.

The flavoring agents can also provide a cooling sensation in the mouth. For example, useful cooling agents include menthol, xylitol, menthane, menthone, ketals, menthone ketals, menthone glycerol ketals, substituted p-menthanes, acyclic carboxamides, substituted cyclohexanamides, substituted cyclohaxane carboxamides, substituted ureas and sulfonamides, substituted menthanols, hydroxymethyl and hydroxymethyl derivatives of p-menthane, 2-mercapto-cyclo-decanone, 2-isopropanyl-5-methylcyclohexanol, hydroxycarboxylic acids with 2-6 carbon atoms, cyclohexanamides, menthyl acetate, menthyl lactate, menthyl salicylate, N-2,3-trimethyl-2-isopropyl butanamide (WS-23), N-ethyl-p-menthane-3-carboxamide (WS-3), menthyl succinate, 3,1-methoxypropane 1,2-diol, among others.

D Nutraceuticals, Health Supplements and Probiotics

The microparticles can also contain one or more nutraceuticals, health supplements and probiotics. Exemplary agents include, for example, but are not limited to, phytochemicals, glucosamine, methylsulfonylmethane, chondroitin, ruscus, bromlein, boswellin, carnitine, hydroxycitric acid, chitosan, acetyl-L-carnitine, phosphatidylserine, huperzine-A, S-adenosylmethione, vinceptine, DMAE, lecithins, ginseng, ashwagandha, ipriflavone, NADH, magnesium malate, and D-ribose; minerals, such as, calcium, iodine, magnesium, zinc, iron, selenium, manganese, chromium, copper, cobalt, molybdenum, and phosphorus; vitamins, such as, vitamin C (ascorbic acid), vitamin A, vitamin B3, vitamin D (ergocalciferol), vitamin E (d1-alpha tocopherol), thiamine (vitamin B-1), riboflavin (vitamin B-2), niacin, pyridoxine (vitamin B-6), cyanocobalamin (vitamin B-12), folic acid, biotin, pantothenic Acid, and vitamin K; fatty acids, such as, the omega-3 unsaturated fatty acids (gamma-linoleic acid, eicosapentaenoic acid, docosahexaenoic acid and the like); oils, such as, borage oil, high carotenoid canola oil, flax seed oil and mixtures thereof; nucleic acids, such as, DNA and RNA; essential amino acids, such as, tryptophan, lysine, methionine, phenylalanine, threonine, valine, leucine, and isoleucine; non-essential amino acids, such as, alanine, arginine, aspartic acid, cystine and cysteine, glutamic acid, glutamine, glycine, histidine, proline and hydroxyproline, serine, taurine, tyrosine and the like; enzymes, such as, bromelain, papain, amylase, cellulase, and coenzyme Q; microorganisms (for example, bacteria, such as, Lactobacillus rhamnosus, Lactobacillus acidophilus, Lactobacillus bulgaricus, Lactobacillus bifidus, Lactobacillus plantarum, and Streptococcus faecium; algae from sources, such as, Spirulina and Chlorella); antioxidants and phytochemicals, such as, anthocyanosides, carotenoids, bioflavonoids, glutathione, catechins, isoflavones, lycopene, ginsenosides, pycnogenol, alkaloids, pygeum phytosterols, sulforaphone, resveratrol, and grape seed extract and foods containing stanol esters, such as benecol; soy products; lecithin; and botanicals, such as herb extracts, and plant extracts.

E—Cosmetic Agents

The microparticles can also contain one or more cosmetic agents. Exemplary cosmetic agents include, for example, moisturizers, emollients, fillers, colorants, perfumes or fragrances, skin conditioners and softeners (for example, urea), vitamins, photoprotectants (for example, sunscreens, such as p-dimethylaminobenzoic acid or glyceryl p-aminobenzoate), antiperspirants, antioxidants, anti-wrinkle materials, as well as any other materials suitable for topical applications; keratolytic agents, such as, salicylic acid; acne treating agents, such as, benzoyl peroxide or sulfur; perfumes, and the like, benzyl alcohol, disodium EDTA, hydroxylated lecithin, alkoxylated diester, polysorbate 80, polysorbate 65, and procaine hydrochloride.

F—Agricultural Agents

The microparticles can also contain one or more agricultural agents. Exemplary agricultural agents include, for example, (1) pesticides, such as, growth regulators, photosynthesis inhibitors, pigment inhibitors, mitotic disrupters, lipid biosynthesis inhibitors, cell wall inhibitors, and cell membrane disrupters; (2) insecticides, such as, phosphoric esters such as azinphos-ethyl, azinphosmethyl, 1-(4-chlorophenyl)-4-(O-ethyl, S-propyl)-phosphoryloxypyrazole, chloropyrifos, coumaphos, demeton, demeton-S-methyl, diazinone, dichlorvos, dimethoate, ethoprophos, etrimfos, fenitrothion, penthion, heptenophos, parathion, parathion-methyl, phosalone, phoxim, pirimiphos-ethyl, pirimiphos-methyl, profenofos, prothiofos, sulfprofos, triazophos and trichlorophone; (3) carbamates, such as, aldicarb, bendiocarb, 2-(1-methylpropyl)phenyl methyl carbamate, butocarboxim, butoxycarboxim, carbaryl, carbofuran, carbosulfan, cloethocarb, isoprocarb, methomyl, oxamyl, pirimicarb, promecarb, propoxur and thiodicarb; (4) pyrethroids, such as, allethrin, alphamethrin, bioresmethrin, byfenthrin, cycloprothrin, cyfluthrin, decamethrin, cyhalothrin, cypermethrin, deltamethrin, alpha-cyano-3-phenyl-2-methylbenzyl 2,2-dimethyl-3-(2-chloro-2-trifluoro-methylvinyl)cyclopropanecarboxylate, fenpropathrin, fenfluthrin, fenvalerate, flucythrinate, flumethrin, fluvalinate, permethrin, resmethrin and tralomethrin; (5) nitroimines and nitromethylenes, such as, 1-[(6-chloro-3-pyridinyl)-methyl]-4,5-dihydro-N-nitro-1H-imidazol-2-amine-(imidacloprid); (6) herbicides, such as, alkanolamine salts of dinitro-o-sec-butylphenol, propylene glycol butyl ethers of 2-(2,4,5-trichlorophenoxy)propanolic acid, chlorinated phenoxy acetic acid and salts or esters thereof, salts of 4-amino-3,5,6-dichloropicalinic acid; (7) fungicides, such as, 3a,4,7,7a-tetrahydro-2-[(trichloromethyl)thio]-1-H-isoindole-1,3 (2H)-dione, 3a,4,7,7a-tetrahydro-2-[(1,1,2,2-tetrachloro-ethyl)thio]-1-H-isoindole-1,3(2H)-dione, 2,4,5,6-tetrachloro-1,3-benzenedicarbonitrile, and sodium methyl-dithiocarbate; (8) agricultural petroleum distillates including light mineral oils and napthalates, and the like.

The amount of active agent or agents included in the microparticles will depend upon the intended use of the microparticles. It is understood that choice of the agent or agents and the amount of agent or agents included within the microparticles is within the level of skill in the art.

It is understood that the microparticles of the invention can be used to deliver a microorganism, for example, bacteria, to a subject. The microorganism can still be viable. In certain embodiments, it is possible to adjust the conditions, for example, the spray drying conditions, so that at least 50%, 60%, 70%, 80%, 90%, or 95% of the microorganisms remain viable after spray drying.

The invention will now be illustrated by means of the following examples which are given for the purpose of illustration only and without any intention to limit the scope of the present invention.

### EXAMPLES

### Example 1

### Microparticles Containing Tramadol or Caffeine

This example describes the synthesis and characterization of microparticles containing 16.5% (w/w) tramadol or 16.5% (w/w) caffeine.

The microparticles were produced as follows. CONTRAMID® excipient obtained from Labopharm, Inc. (Laval, Canada) was dispersed in 0.2 M phosphate buffer pH 6.8-8 by high shear mixing (1800 rpm) using a MP550 Turbo mixer (Robot® Coupe, Jackson, Miss.) at 40-45° C. for approximately 15 minutes to give a final dispersion having a concentration of 10% (w/w). KOLLIDON VA-64 Fine obtained from BASF (New Jersey) was solubilised in water, at room temperature, by vigorous stirring using a stirring bar for 10 minutes to give a final concentration of KOLLIDON VA-64 Fine of 20% (w/w). The active ingredient, for example, tramadol HCl (see TABLE 1) or caffeine (see TABLE 2) then was added to the aqueous solution of KOLLIDON VA-64 Fine to give the appropriate drug loading. The solution containing KOLLIDON VA-64 Fine and the active ingredient then was added gradually (over 20-25 minutes) to the solution containing the CONTRAMID excipient by high shear mixing (1800 rpm) using a MP550 Turbo mixer (Robot® Coupe, Jackson, Miss.) at room temperature. The resulting mixture was maintained under magnetic stirring over night ready for spray drying.

The resulting mixture then was spray dried in a mini Spray Dryer B-290 (BÜCHI Labortechnik AG, Switzerland) operating with co-current air and product stream. The air introduced via the air inlet had a temperature of 190° C. The outlet temperature and pressure were 85-90° C. and 12 bar, respectively. The resulting microparticles were collected and characterized.

The composition of the solution spray dried to produce the tramadol-containing particles is described in TABLE 1, where the ratio of cross-linked high amylose starch (CONTRAMID):KOLLIDON VA-64 Fine was about 60:40.

TABLE 1

| Composition | Weight (g) |
|---|---|
| CONTRAMID | 46.8 |
| KOLLIDON VA-64 Fine | 31.3 |
| Tramadol HCl | 15.5 |
| 0.2 M Phosphate buffer | 420.8 |
| Water | 124.6 |

After spray drying, it is contemplated that the ratio of the cross-linked high amylose starch:KOLLIDON VA-64 Fine is about 60:40.

The composition of the solution spray dried to produce the caffeine-containing particles is described in TABLE 2, where the ratio of cross-linked high amylose starch (CONTRAMID):KOLLIDON VA-64 Fine was about 60:40.

TABLE 2

| Composition | Weight (g) |
|---|---|
| CONTRAMID | 46.8 |
| KOLLIDON VA-64 Fine | 31.3 |
| Caffeine | 15.5 |
| 0.2 M Phosphate buffer | 420.8 |
| Water | 124.6 |

After spray drying, it is contemplated that the ratio of the cross-linked high-amylose starch:KOLLIDON VA-64 Fine is about 60:40.

The resulting microparticles containing either tramadol or caffeine then were characterized by scanning electron microscopy (SEM) (JSM-840, JEOL, Tokyo, Japan) for microstructure characterization. Powder specimens of microspheres were fixed on the sample stubs with liquid adhesive, and then coated with a thin layer of gold using an ion sputter. The coated powder then was examined by SEM at an accelerating voltage of 15 kV. The SEM pictures were taken and visually analyzed for the microstructural properties of the spray-dried microencapsulated products.

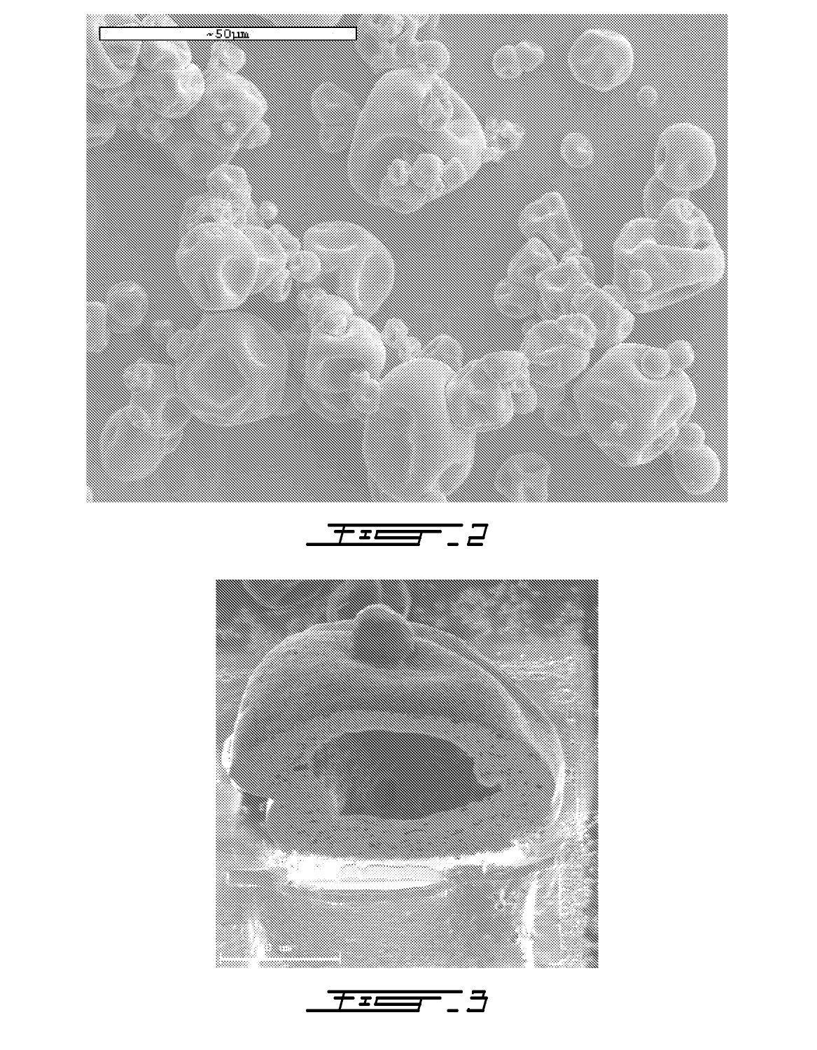

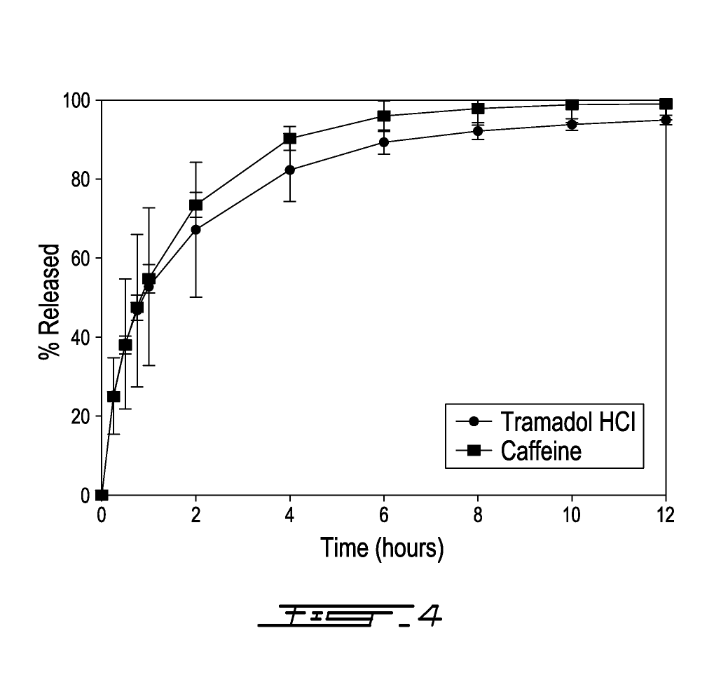

The resulting tramadol and caffeine containing microparticles had a mean diameter of about 1 μm to about 200 μm, with the majority (i.e., greater than 50%) of the particles having a mean diameter in the range of from about 5 μm to about 50 μm. An image of exemplary tramadol containing microparticles taken by SEM is shown in FIG. 2. A cross-sectional view of exemplary tramadol containing microparticles is shown in FIG. 3, which demonstrates that the microparticles were hollow.

The microparticles had a loading efficiency for both tramadol and caffeine of greater than 90%. The final concentrations of the tramadol HCl and caffeine were about 16% (w/w).

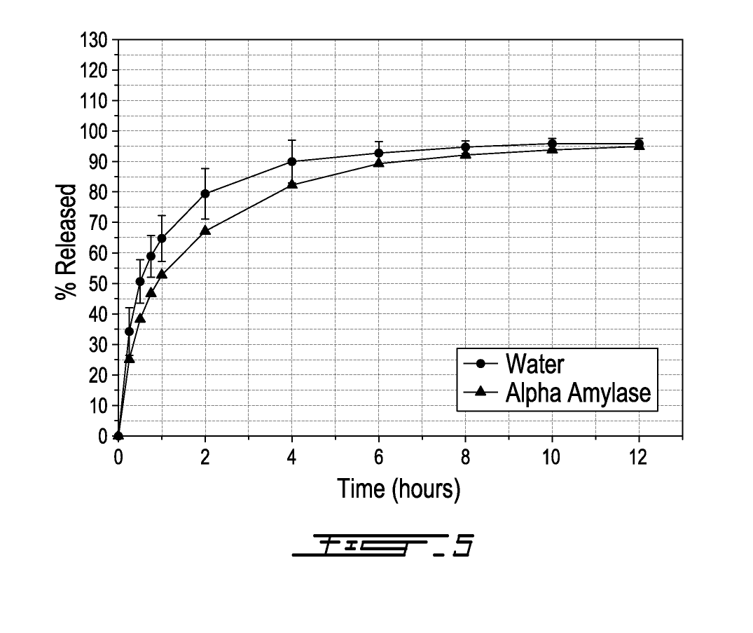

In addition, the release kinetics of the active agents from the microparticles were measured by a dissolution tester (VANKEL 10-1600, VK 700 from Varian) at a stirring speed of 100 rpm. Three 1.5 g samples from each batch of spray dried microparticles were tested using 900 mL of dissolution medium (water) maintained at 37° C. An aliquot of the release medium (5 mL) was withdrawn at the following time intervals (0.25, 0.5, 0.75, 1, 2, 4, 6, 8, 10 and 12 hours) and then analyzed. An equivalent amount of fresh dissolution medium, which was prewarmed at 37° C. was replaced in the 900 mL dissolution bath. The collected samples then were analyzed for tramadol or caffeine content by HPLC. The results are summarized in FIG. 4

FIG. 4 shows that both the tramadol and the caffeine were released with similar release kinetics with about 50-60% of the active ingredient being released by 2 hours, about 60-80% of the active ingredient being released by 3 hours, about 70-90% of the active ingredient being released by 4 hours, greater than about 80% of the active ingredient being released by 6 hours, and greater than about 90% of the active ingredient being released by 8 hours.

Drug release in the presence/absence of α-amylase was measured using the same dissolution tester (VANKEL 10-1600, VK 700) at a stirring speed of 100 rpm. Three 1.5 g samples of microparticles from each batch of spray dried microparticles were tested using 900 mL of dissolution medium (three baths with water and three other baths containing 5000 Units/L α-amylase) at 37° C. An aliquot of the release medium (5 mL) was withdrawn at the following time intervals (0.25, 0.5, 0.75, 1, 2, 4, 6, 8, 10 and 12 hours) and then analyzed. An equivalent amount of fresh dissolution medium, which was prewarmed at 37° C. was replaced in the 900 mL dissolution bath. Collected samples then were analyzed for tramadol content by HPLC.

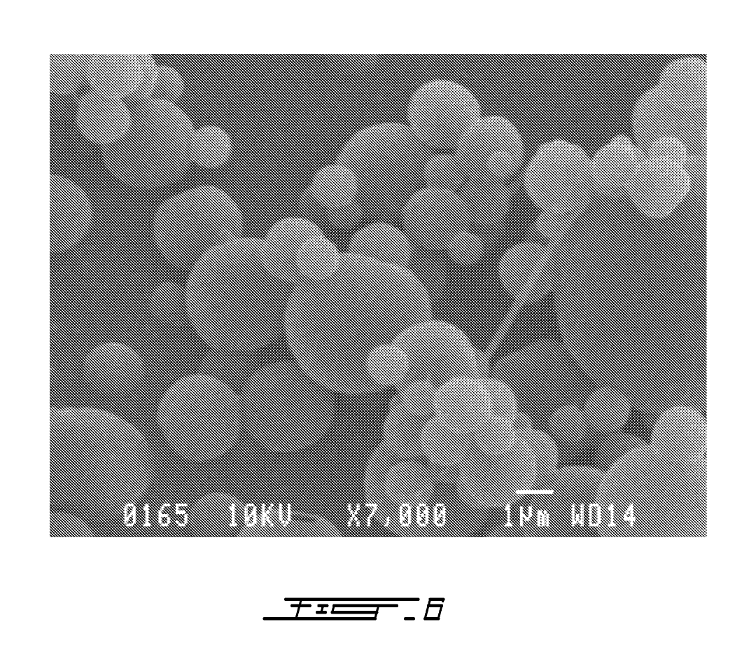

The results shown in FIG. 5 show that the microparticles of the invention are substantially resistant to α-amylase as the release profiles in both water and water containing α-amylase were substantially the same. The rate of tramadol release from the microparticles, if anything, was slightly slower in the presence of amylase versus than in the absence of amylase.

### Example 2

### Tramadol-Containing Microparticles with a High Loading Capacity

This example describes the synthesis and characterization of microparticles containing 50% (w/w) tramadol.

The microparticles containing tramadol were produced essentially as described in Example 1 except for the differing amounts of each of the initial ingredients that were combined prior to spray drying and the mixture also omitted KOLLIDON VA-64 Fine. The weight of the various starting materials are set forth in TABLE 3.

TABLE 3

| Composition | Weight (g) |
|---|---|
| CONTRAMID | 1,038.5 |
| Tramadol HCl | 1,038.5 |
| 0.2 M Phosphate buffer | 446.5 |
| Water | 15,303.5 |

CONTRAMID® excipient obtained from Labopharm, Inc. (Laval, Canada) was dispersed in phosphate buffer pH 6.8-8 by high shear mixing (1800 rpm) using the Robot Coupe MP 550 Turbo mixer at about 55° C. for approximately 15 minutes to give a mixture containing 10% (w/w) CONTRAMID® excipient. Tramadol then was added to the CONTRAMID® excipient-based dispersion under stirring. The mixture was stirred overnight at room temperature to allow for complete hydration.

On the following day, the CONTRAMID® excipient-based dispersion containing tramadol was heated to 55° C. before being added to the spray dryer.

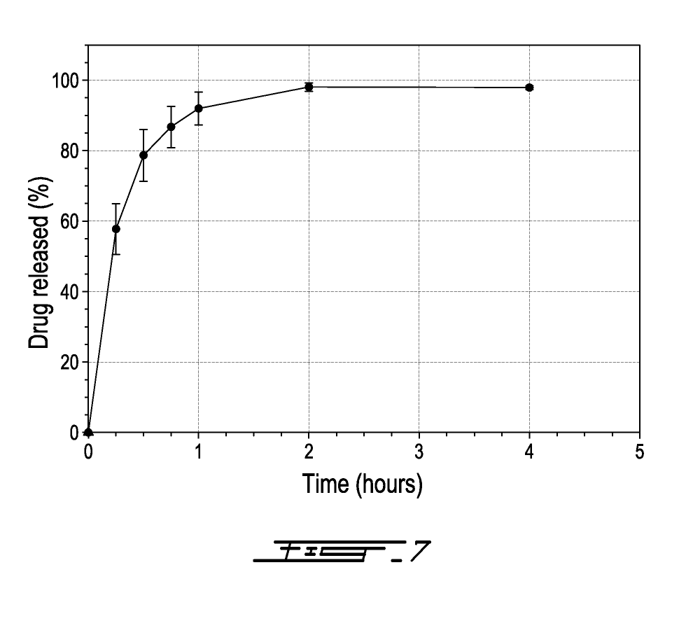

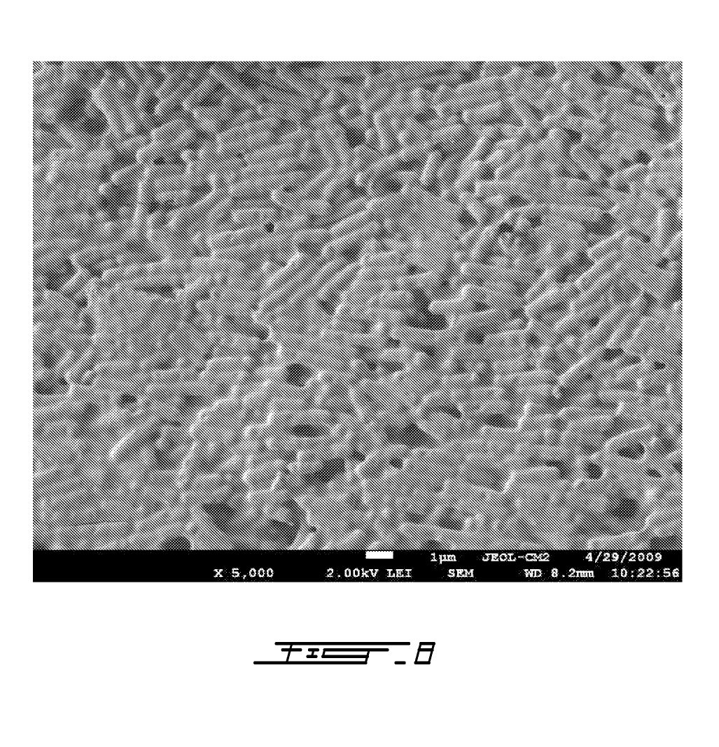

The resulting microparticles were characterized by SEM, the results of which are shown in FIG. 6. Furthermore, the in vitro release kinetics were measured as described in Example 1 (the dissolution medium was amylase free water). The results are shown in FIG. 7, wherein from about 70% to about 90% of the tramadol was released in 30 minutes, from about 80% to about 95% of the tramadol was released in 1 hour, and from about 90% to about 100% of the tramadol was released in 2 hours.

### Example 3

### Microparticles Containing Agents with Low Melting Point

This example describes a method of incorporating a water insoluble agent with a low melting point, for example, menthol, into microparticles.

CONTRAMID® excipient from Labopharm, Inc. (Laval, Canada) was dispersed in phosphate buffer pH 6.8-8 by high shear mixing (1800 rpm) using a MP550 Turbo mixer (Robot® Coupe, Jackson, Miss.) at about 55° C. for approximately 15 minutes to give a mixture containing 10% (w/w) CONTRAMID® excipient. The mixture was stirred overnight at room temperature to permit for complete hydration.

On the following day, the active agent (menthol, melting point about 42° C.) was melted by heating to about 55° C. and then was added to the CONTRAMID® excipient mixture and further mixed for 5 to 10 minutes using the Robot® Coupe MP550 Turbo mixer (12,000 rpm) (Robot® Coupe, Jackson, Miss.) to form a milky white emulsion. Optionally, a surface active agent, such as, Tween® 20 or Tween® 80, could be added to improve the stability of the emulsion. The emulsion then was continuously mixed at about 50° C. to 70° C. before being dried by a spray dryer, as described in Example 1.

### Example 4

### Microparticles Containing Agents with High Melting Point

This example describes the method of incorporating a water insoluble agent with a high melting point, such as vanillin, into microparticles.

CONTRAMID® excipient obtained from Labopharm, Inc. (Laval, Canada) was dispersed in phosphate buffer pH 6.8-8 by high shear mixing (1800 rpm) using a MP550 Turbo mixer (Robot® Coupe, Jackson, Miss.) at about 55° C. for approximately 15 minutes to give a mixture containing 10% (w/w) CONTRAMID® excipient. The mixture was stirred overnight at room temperature to allow for complete hydration.

On the following day, the active agent (vanillin, melting point about 85° C.) was dissolved in a suitable dispersing agent (namely, mineral oil or castor oil), and heated to 55° C. The oily solution then was added to the CONTRAMID® excipient mixture by high shear mixing (12,000 rpm) using a MP550 turbo mixer (Robot® Coupe, Jackson, Miss.) to form a milky white emulsion. Optionally, a surface active agent, such as, Tween® 20 or Tween® 80, could be added to the emulsion in order to improve the stability of the emulsion. The emulsion was continuously mixed at about 70° C. before being spray dried, as described in Example 1.

### Example 5

### Contramid® Microparticles Containing a Probiotic Agent

This example describes the preparation and characterization of Contramid® excipient-based microparticles containing a probiotic agent, Lactobacillus rhamnosus HA-111, at concentration level of 8.5×10⁹ cfu/mL.

Lactobacillus rhamnosus is a probiotic bacterium that can slow or inhibit the growth of harmful bacteria in the intestine. It is often used as a natural preservative in yogurt and other dairy products to extend shelf life.

Microparticles containing the probiotic agent were produced essentially as described in Example 1. The CONTRAMID® excipient was first dispersed in phosphate buffer pH 6.8-8 by high shear mixing (1800 rpm) using a MP550 Turbo mixer (Robot® Coupe, Jackson, Miss.) at 40-45° C. for approximately 15 minutes to give a final dispersion having a concentration of 10% (w/w). KOLLIDON VA-64 Fine obtained from BASF (New Jersey) was solubilised in water, at room temperature, by vigorous stirring for 10 minutes to give a final concentration of KOLLIDON VA-64 Fine of 20% (w/w). The KOLLIDON VA-64 solution then was mixed with the CONTRAMID® dispersion and the dispersion then was cooled to a temperature in the range of 6° C.-12° C. by circulating cold water (4° C.) through the jacket of the container. The probiotic suspension then was added to the CONTRAMID® based dispersion under low agitation. The weight of the various starting materials is set forth in TABLE 4.

TABLE 4

| Composition | Weight (g) |
|---|---|
| CONTRAMID ® | 960 |
| KOLLIDON VA-64 | 400 |
| Lactobacillus rhamnosus HA-111 | 9000 |
| suspension (8.5 × 10⁹ cfu/mL) | |
| 0.2 M Phosphate buffer | 347 |
| Water | 11893 |

The dispersion was continuously mixed at about 7° C. before spray drying, as described in Example 1. The air inlet temperature was 180° C., and the air outlet temperature was either 60° C. or 80° C.

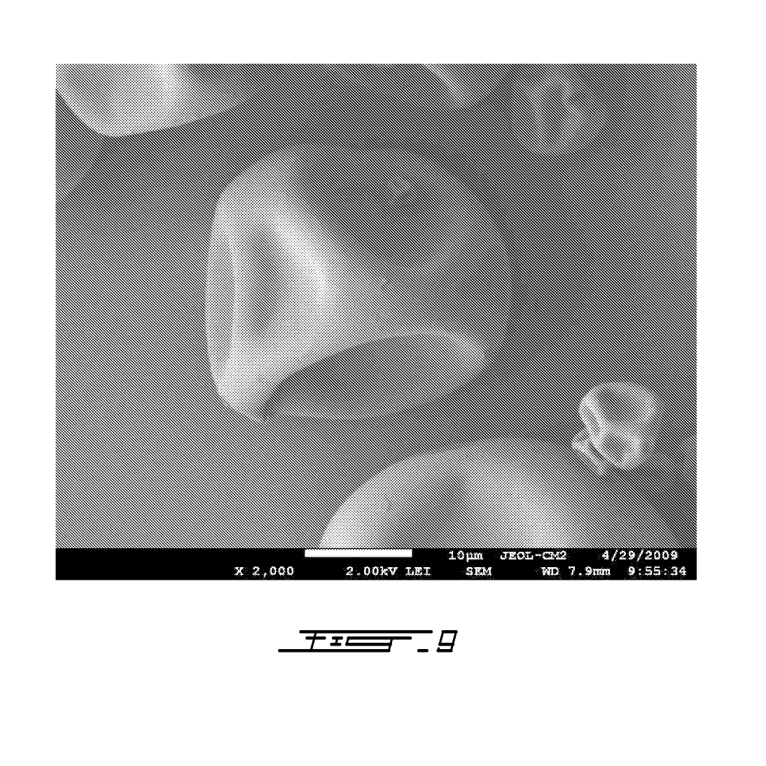

An image of the bacterial strain Lactobacillus rhamnosus taken by SEM is shown in FIG. 8, and an image of the resulting spray dried microparticles also taken by SEM is shown in FIG. 9. It is believed that the bacteria were encapsulated into CONTRAMID® excipient-based microparticles after spray drying in view of the absence of rods in FIG. 9.

The yield (colony forming units (cfu)) of surviving bacteria present in the microparticles after spray drying is summarized in Table 5.

TABLE 5

| Air outlet | Initial cfu before | cfu after | bacterial survival |
|---|---|---|---|
| temperature (° C.) | spray drying | spray drying | (%) after drying |
| 60° C. | 8.5 × 10⁹ | 8.5 × 10⁹ | ≧95 |
| 80° C. | 8.5 × 10⁹ | 2 × 10⁹ | 23.5 |

From TABLE 5, the survival rate of Lactobacillus rhamnosus after spray drying was greater than 95% at 60° C. and about 23.5% at 80° C.

It is contemplated that other bacterial strains can be introduced into the microparticles according to the invention, for example, Lactobacillus acidophilus, Lactobacillus bulgaricus, Lactobacillus plantarum, Staphococcus faecium, and the like.

### Example 6

### CONTRAMID® Microparticles Containing a Herbal Products

This example describes the preparation and characterization of CONTRAMID® excipient-based microparticles containing a herbal product.

The microparticles containing a blend of plant extracts were produced essentially as described in Example 1. The CONTRAMID® excipient was first dispersed in 0.2 M phosphate buffer pH 6.8-8 by high shear mixing (1800 rpm) using a MP550 Turbo mixer (Robot® Coupe, Jackson, Miss.) at 40-45° C. for approximately 15 minutes to give a final dispersion having a concentration of 10% (w/w). A blend of herbal extracts was suspended in 0.2 M phosphate buffer, pH 6.8 containing 0.25% (w/v) soy lecithin and maintained under mild agitation overnight to avoid sedimentation of particles. The herbal blend suspension then was added to the CONTRAMID® dispersion under agitation.

The weight of the various starting materials is set forth in TABLE 6.

TABLE 6

| Composition | Weight (g) |
|---|---|
| CONTRAMID ® | 750 |
| Soy lecithin | 10 |
| Herbal blend | 750 |
| 0.2 M Phosphate buffer | 333 |
| Water | 11417 |

The dispersion was continuously mixed at room temperature before being spray dried, as described in Example 1. The air inlet temperature was 190° C., and the air outlet temperature was 70° C.

The resulting spray dried microparticles resulted in a fine powder that was readily dispersible in water as opposed to initial herbal blend that was not readily dispersible in water. The microparticles also resulted in significant taste and odor masking of the initial herbal blend making it more palatable for consumption.

### INCORPORATION BY REFERENCE

The entire disclosure of each of the patent and scientific documents referred to herein is incorporated by reference for all purposes.

### EQUIVALENTS

Although the present invention has been illustrated by means of preferred embodiments thereof, it is understood that the invention intends to cover broad aspects thereof without departing from the spirit and scope of the invention as defined in the appended claims.

1. A method of producing microparticles that stabilize and/or control the release of an agent disposed therein, the method comprising
(a) providing a solution comprising a mixture of from about 5% w/w to about 20% w/w of cross-linked high amylose starch and the agent to be released; and
(b) spray drying the mixture of step (a) in a spray dryer to produce microparticles having a mean diameter of from about 1 μm to about 200 μm.

2. The method of claim 1, wherein the microparticles have a mean diameter in the range from about 5 μm to about 200 μm, from about 5 μm to about 150 μm, from about 1 μm to about 100 μm, from about 10 μm to about 100 μm, from about 1 μm to about 50 μm, from about 1 μm to about 40 μm, from about 1 μm to about 30 μm, from about 2 μm to about 50 μm, from about 3 μm to about 50 μm, from about 4 μm to about 50 μm, or from about 5 μm to about 50 μm.

3. The method of claim 1, wherein the mixture provided in step (a) comprises from about 7% w/w to about 15% w/w of cross-linked high amylose starch.

4. The method of claim 1, wherein the agent comprises from about 5% to about 50% (w/w) of the microparticles produced in step (b).

5. The method of claim 4, wherein the agent to be released comprises from about 10% to about 45% (w/w), from about 15% to about 40% (w/w), from about 15% to about 35% (w/w) or from about 20% to about 35% (w/w) of the microparticles produced in step (b).

6. The method of claim 1, wherein the mixture provided in step (a) is created by mixing the solution comprising the cross-linked high amylose starch and the agent at a temperature in the range of from about 15° C. to about 70° C.

7. The method of claim 6, wherein the temperature is in the range of from about 30° C. to about 60° C.

8. The method of claim 1, wherein, in step (a), the agent is melted prior to mixing with the cross-linked high amylose starch.

9. The method of any claim 1, wherein, in step (a), the agent is mixed with a dispersing agent prior to mixing with the cross-linked high amylose starch.

10. The method of claim 8, wherein, in step (a), the solution further comprises a surface active agent.

11. (canceled)

12. The method of claim 1, wherein, in step (a), the solution further comprises a viscosity reducing agent.

13-14. (canceled)

15. The method of claim 12, wherein the viscosity reducing agent is a polyvinylpyrrolidone-vinyl acetate copolymer.

16. The method of claim 12, wherein the ratio of the cross-linked high amylose starch to the viscosity reducing agent is from about 80:20 (w/w) to about 40:60 (w/w).

17. The method of claim 16, wherein the ratio of the cross-linked high amylose starch to the viscosity reducing agent is about 60:40 (w/w).

18. The method of claim 1, wherein the mixture provided in step (a) is substantially free of pectin.

19. The method of claim 1, wherein, in step (b), the spray dryer has an air inlet temperature in the range of from about 125° C. to about 250° C. and an air outlet temperature in the range of from about 50° C. to about 100° C.

20. The method of claim 1, wherein the agent is a pharmaceutical, a taste masking agent, a flavoring agent, or a microorganism.

21.-23. (canceled)

24. The method of claim 20, wherein the microorganism is a bacteria.

25. The method of claim 24, wherein at least 50% of the bacteria remain viable after spray drying.

26. A composition of microparticles produced by the method of claim 1.

27. A microparticle composition comprising:
a plurality of microparticles comprising from about 35% (w/w) to about 70% (w/w) of cross-linked high amylose starch and an agent for release from the microparticles, wherein the microparticles have a mean diameter in the range from about 1 μm to about 200 μm.

28. The composition of claim 27, wherein the microparticles have a mean diameter in the range from about 5 μm to about 200 μm, from about 5 μm to about 150 μm, from about 1 μm to about 100 μm, from about 10 μm to about 100 μm, from about 1 μm to about 50 μm, from about 1 μm to about 40 μm, from about 1 μm to about 30 μm, from about 2 μm to about 50 μm, from about 3 μm to about 50 μm, from about 4 μm to about 50 μm, or from about 5 μm to about 50 μm.

29. The composition of claim 27, wherein the microparticles further comprise a viscosity reducing agent.

30.-31. (canceled)

32. The composition of claim 27, wherein the microparticles are substantially free of pectin.

33. The composition of claim 27, wherein the microparticles are substantially resistant to degradation by α-amylase.

34. The composition of claim 27, wherein the agent comprises from about 5% to about 50% (w/w) of the microparticles.

35. The composition of claim 34, wherein the agent comprises from about 10% to about 45% (w/w), from about 15% to about 40% (w/w), from about 15% to about 35% (w/w), or from about 20% to about 35% (w/w) of the microparticles.

36. The composition of claim 27, wherein the agent is a pharmaceutical.

37. A formulation for delivery of an agent, wherein the formulation comprises the microparticles of claim 27.

38. The formulation of claim 37, wherein the microparticles are disposed within a tablet, caplet, chewing gum, film, wafer or an orally disintegrating tablet.

39. A method of providing controlled release of an agent, the method comprising orally administering to a subject the formulation of claim 37.

40. The method of claim 1, wherein the mixture spray dried in step (b) is an emulsion.
